(19) United States
(12) Patent Application Publication
(10) Pub. No.: US 2004/0171627 A1
(43) Pub. Date: Sep. 2, 2004

(54) Treatment of renal fibrosis

(75) Inventors: Arnon Nagler, Jerusalem (IL); Israel Vlodavsky, Mevaseret Zion (IL); Mark Pines, Rehovot (IL); Shai Yarkoni, Kfar Saba (IL)

Correspondence Address: WINSTON & STRAWN, PATENT DEPARTMENT, 1400 L STREET, N.W., WASHINGTON, DC, 20005-3502, US

(73) Assignees: Hadasit; State Of Israel; Collgard

(21) Appl. No.: 10/723,918

(22) Filed: Nov. 24, 2003

Related U.S. Application Data
(63) Continuation of application No. PCT/IL02/00408, filed on May 23, 2002.

(30) Foreign Application Priority Data
May 24, 2001 (IL) IL143366

(51) Int. Cl. A61K 31/517

(52) U.S. Cl. 514/266.22

(57) ABSTRACT

The present invention relates to compositions containing quinazolinones. More particularly, the present invention relates to a composition for the treatment of renal fibrosis. This composition includes, as an active ingredient, a quinazolinone derivative such as halofurginone, which is shown herein to slow or prevent progression of renal fibrosis in vivo thereby mitigating or preventing end-stage renal failure.

### CROSS-REFERENCE TO RELATED APPLICATIONS

This application is a continuation of International application PCT/IL02/00408 filed May 23, 2002, the entire content of which is expressly incorporated herein by reference thereto.

### FIELD OF THE INVENTION

The present invention relates to compositions containing quinazolinones. More particularly, the present invention relates to compositions for treatment renal fibrosis, comprising as an active ingredient therein a quinazolinone derivative as herein defined.

### BACKGROUND OF THE INVENTION

Halofuginone

U.S. Pat. No. 3,320,124 discloses and claimed a method for treating coccidiosis with quinazolinone derivatives. Halofuginone, otherwise known as 7-bromo-6-chloro-3-[3-(3-hydroxy-2-piperidinyl)-2-oxopropyl]-4(3H)-quinazolinone (one of the quinazolinone derivative), was first described and claimed in said patent by American Cyanamid, and was the preferred compound taught by said patent and the one commercialized from among derivatives described and claimed therein. Subsequently, U.S. Pat. Nos. 4,824,847; 4,855,299; 4,861,758 and 5,215,993 all relate to the coccidiocidal properties of halofuginone.

More recently, it was disclosed in U.S. Pat. No. 5,449,678 that these quinazolinone derivatives are unexpectedly useful for the treatment of a fibrotic condition. This disclosure provides compositions of a specific inhibitor comprising a therapeutically effective amount of a pharmaceutically active compound of the formula:

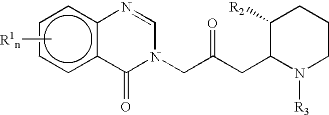

wherein: n=1-2

R₁ is a member of the group consisting of hydrogen, halogen, nitro, benzo, lower alkyl, phenyl and lower alkoxy;

R₂ is a member of the group consisting of hydroxy, acetoxy and lower alkoxy; and R₃ is a member of the group consisting of hydrogen and lower alkenoxy-carbonyl. Pharmaceutically acceptable salts thereof are also included. Of this group of compounds, halofuginone has been found to be particularly effective for the disclosed treatment.

U.S. Pat. No. 5,449,678 discloses that the aforementioned compounds are effective in the treatment of fibrotic conditions such as scleroderma and graft-versus-host disease (GVHD). U.S. Pat. No. 5,891,879 further discloses that these compounds are effective in treating restenosis. The two former conditions are associated with excessive collagen deposition, which can be inhibited by halofuginone. Restenosis is characterized by smooth muscle cell proliferation and extracellular matrix accumulation within the lumen of affected blood vessels in response to a vascular injury [Choi et al., Arch. Surg., 130:257-261, 1995]. One hallmark of such smooth muscle cell proliferation is a phenotypic alteration, from the normal contractile phenotype to a synthetic one. Type I collagen has been shown to support such a phenotypic alteration, which can be blocked by halofuginone [Choi et al., Arch. Surg., 130: 257-261, 1995; U.S. Pat. No. 5,449,678].

Notably, the in vitro action of halofuginone does not always predict its in vivo effects. For example, as demonstrated in U.S. Pat. No. 5,449,678, halofuginone inhibits the synthesis of collagen type I in bone chrondrocytes in vitro. However, chickens treated with halofuginone were not reported to have an increased rate of bone breakage, indicating that the effect is not seen in vivo.

In addition, even though halofuginone inhibits collagen synthesis by fibroblasts in vitro, it promotes wound healing in vivo (WO 01/17531). Thus, the exact behavior of halofuginone in vivo cannot always be accurately predicted from in vitro studies.

Chronic Renal Failure

The progression of chronic renal failure (CRF) represents one of the most challenging problems in nephrology, as it leads to a large number of patients reaching end-stage renal failure requiring long-term dialysis treatment. Many renal diseases progress to end-stage renal failure with glomerular sclerosis and/or medullar fibrosis, independent of the initial pathogenic mechanism. This suggests that various progressive renal diseases may exhibit a common destructive pathway that leads to focal and eventually diffuse glomerulosclerosis and chronic tubuloinsterstitial disease.

Since there is a possibility that direct inhibition of renal fibrosis, considered as the final common pathway, will attenuate the development of chronic renal failure (CRF), therapeutic antifibrotic strategies should be targeted to reduce or eliminate this process.

Chronic kidney diseases are characterized by the accumulation of extracellular matrix (ECM) in glomeruli and interstitium, which lead finally to renal fibrosis and chronic renal failure [Klahr S. et al., N Engl J Med 318:1657-1666,1988]. Glomerular sclerosis is characterized by replacement of the functional glomeruli by connective tissue mainly through expansion of the mesangial cells and deposition of ECM. Fibrosis is believed to result from excessive synthesis of ECM and a concomitant decrease in its breakdown.

The pathogenesis of renal fibrosis includes the formation of fibrotic tissue in the kidney. The formation of fibrotic tissue is characterized by the deposition of abnormally large amounts of collagen. Following kidney injury (the term “injury” includes physical, toxic and vascular injuries) mesangial cells have the capacity to synthesize collagen types I and III, as opposed to the exclusive presence of type IV collagen in healthy glomeruli (Trai et al., 1994). In vitro, mesangial cells have the capacity to release matrix metallo-proteinase (MMPr) capable of degrading collagen IV, but not collagen I and III (Daniel et. al. 1998). The synthesis of collagen is also involved in a number of other pathological conditions. For example, clinical conditions and disorders associated with primary or secondary fibrosis, such as systemic sclerosis, graft-versus-host disease (GVHD), pulmonary and hepatic fibrosis and a large variety of autoimmune disorders, are distinguished by excessive production of connective tissue, which results in the destruction of normal tissue architecture and function. These diseases can best be interpreted in terms of perturbations in cellular functions, a major manifestation of which is excessive collagen synthesis and deposition. The crucial role of collagen in fibrosis has prompted attempts to develop drugs that inhibit its accumulation [K. I. Kivirikko, Annals of Medicine, Vol. 25, pp. 113-126 (1993)].

Interstitial fibrosis is characterized by the destruction of renal tubules and interstitial capillaries as well as by the accumulation of extracellular matrix proteins [M. Fukagawa et. al. Nephrol Dial Transplant (1999) 14:2793-2795].

Focal and segmental glomerulosclerosis (FSGS) is the histological description of a form of glomerular injury that is usually associated with proteinuria and progressive loss of renal function [see H. G. Rennke and P. S. Klein, “Pathogenesis and Significance of nonprimary Focal and segmental Glomerulosclerosis” Am. J. Kid. Dis. Vol. 13, pp. 443-46 (1989)].

Originally, FSGS was described in nephrotic patients who had died with end-stage renal failure. In more recent years, FSGS has been identified as a final common pathway in the glomerulus in a number of human systemic and renal diseases. These include processes such as normal aging and diabetic nephropathy. The pathologic lesion of FSGS can result from a variety of seemingly unrelated injurious stimuli, leading through extracellular matrix deposition and glomerulosclerosis to renal demise long after the termination of the initial injury.

Such drugs can act by modulating the synthesis of the procollagen polypeptide chains, or by inhibiting specific post-translational events, which will lead either to reduced formation of extra-cellular collagen fibers or to an accumulation of fibers with altered properties. Unfortunately, only a few inhibitors of collagen synthesis are available, despite the importance of this protein in sustaining tissue integrity and its involvement in various disorders.

For example, cytotoxic drugs have been used in an attempt to slow the proliferation of collagen-producing fibroblasts [J. A. Casas, et al., Ann. Rhem. Dis., 46: 763, 1987], such as colchicine, which slows collagen secretion into the extracellular matrix [D. Kershenobich, et al., N. Engl. J. Med., 318:1709, 1988], as well as inhibitors of key collagen metabolism enzymes [K. Karvonen, et al., J. Biol. Chem., 265: 8414, 1990; C. J. Cunliffe, et al., J. Med. Chem., 35:2652, 1992].

Unfortunately, none of these inhibitors are collagen-type specific. Also, there are serious concerns about the toxic consequences of interfering with biosynthesis of other vital collagenous molecules, such as Clq in the classical complement pathway, acetylcholine esterase of the neuro-muscular junction endplate, conglutinin and renal surfactant apoprotein.

Other drugs that can inhibit collagen synthesis, such as nifedipine and phenyloin, inhibit synthesis of other proteins as well, thereby non-specifically blocking the collagen biosynthetic pathway [T. Salo, et al., J. Oral Pathol. Med., 19: 404, 1990].

Collagen cross-linking inhibitors, such as β-amino-propionitrile, are also non-specific, although they can serve as useful anti-fibrotic agents. Their prolonged use causes lathritic syndrome and interferes with elastogenesis, since elastin, another fibrous connective tissue protein, is also cross-linked. In addition, the collagen cross-linking inhibitory effect is secondary, and collagen overproduction has to precede its degradation by collagenase. Thus, a type-specific inhibitor of the synthesis of collagen itself is clearly required as an anti-fibrotic agent.

The ability of halofuginone, or other related quinazolinone derivatives, to block or inhibit pathological processes related to renal fibrosis, has only been shown in U.S. Pat. No. 5,998,442. That patent disclosed a pharmaceutical composition containing quinazolinone derivatives for attenuation of abnormal Mesangial Cell proliferation wherein all the examples were tested in vitro. Moreover, the strong fibrotic process in the tubulointerstitial compartments that characterizes the renal fibrotic diseases does not involve any mesangial cell proliferation.

It is notoriously well known in the art of drug development that pharmacological effects obtained in vitro are not necessarily reproducible in vivo in a living organism. Therefore, it is not possible to extrapolate from the observed inhibition of abnormal mesangial cell proliferation in vitro that these compounds are effective for treatment of kidney disease in which renal fibrosis may be either a cause or a result of some other underlying pathology. It was clearly impossible to anticipate that halofuginone would be useful to prevent progression of renal disease to end-stage renal failure.

Nothing in the prior art taught or suggested that halofuginone would be useful in the treatment of renal fibrosis in vivo. Thus, the ability of halofuginone and related compounds to slow or halt progression of fibrosis in the kidneys is both novel and non obvious.

### SUMMARY OF THE INVENTION

Unexpectedly, it has been found, as described below, that pharmaceutical compositions containing quinazolinone derivatives, especially halofuginone, can also inhibit the pathophysiological processes of renal fibrosis in vivo, including the effect on both the glomeruli and the tubuli interstitial compartments, possibly by inhibiting collagen type I synthesis although other mechanisms can also be responsible. While inhibition of collagen type I synthesis is proposed as one plausible mechanism, it is not desired to be limited to a single mechanism, nor it is necessary since the in vivo data presented below clearly demonstrate the efficacy of halofuginone as an inhibitor of renal fibrosis in vivo.

The present invention provides a composition for treating renal fibrosis, comprising a pharmaceutically effective amount of a compound in combination with a pharmaceutically acceptable carrier, the compound being a member of a group having the general formula:

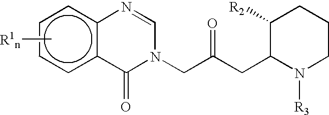

wherein: n=1-2

R₁ is a member of the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl and pharmaceutically acceptable salts thereof.

According to further preferred embodiments of the present invention, the compound is preferably halofuginone.

According to another embodiment the present invention provides a method of manufacturing a medicament for treating renal fibrosis, including the step of placing a pharmaceutically effective amount of a compound in a pharmaceutically acceptable carrier, the compound being a member of a group having the general formula:

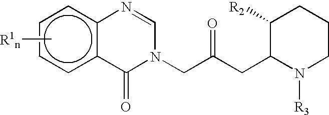

wherein: n=1-2

R₁ is a member of the group consisting of hydrogen, halogen, nitro, benzo, lower alkyl, phenyl and lower alkoxy;

R₂ is a member of the group consisting of hydroxy, acetoxy and lower alkoxy; and R₃ is a member of the group consisting of hydrogen and lower alkenoxy-carbonyl and pharmaceutically acceptable salts thereof.

According to yet another embodiment the present invention provides a method for the treatment of renal fibrosis in a subject including the step of administering a pharmaceutically effective amount of a compound having the general formula:

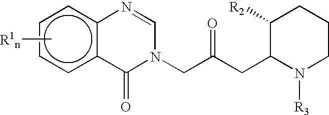

wherein: n=1-2

R₁ is a member of the group consisting of hydrogen, halogen, nitro, benzo, lower alkyl, phenyl and lower alkoxy;

R₂ is a member of the group consisting of hydroxy, acetoxy and lower alkoxy; and R₃ is a member of the group consisting of hydrogen and lower alkenoxy-carbonyl, and pharmaceutically acceptable salts thereof.

The renal fibrosis can be primary or secondary. Primary renal fibrosis is related to a condition that affects the kidney without being the result of some other disease or disorder, whereas secondary renal fibrosis is the result of some underlying pathology.

The secondary condition may be caused by high hypertension, diabetes complications, autoimmune disease, and other disorders.

The present invention further provides a method for preventing renal fibrosis from progressing to end-stage renal failure comprising administering to a subject in need thereof a therapeutically effective amount of compound in a pharmaceutically acceptable carrier, said compound being a member of a group having the general formula:

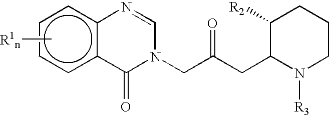

wherein: n=1-2

R₁ is a member of the group consisting of hydrogen, halogen, nitro, benzo, lower alkyl, phenyl and lower alkoxy;

R₂ is a member of the group consisting of hydroxy, acetoxy and lower alkoxy; and R₃ is a member of the group consisting of hydrogen and lower alkenoxy-carbonyl, and pharmaceutically acceptable salts thereof

### BRIEF DESCRIPTION OF THE DRAWINGS

The invention is described herein by way of example only, with reference to the accompanying drawings, wherein:

FIG. 1: The effect of halofuginone on systolic blood pressure (SBP) in rats.

(*) Significantly lower (p<0.01) than both RMR groups

FIG. 2: The effect of halofuginone on protein concentration in rat urine.

(*) Significantly lower (p<0.01) than both RMR groups

FIG. 3: The effect of halofuginone on body weight in rats.

FIG. 4: The effect of halofuginone on creatinine clearance (CCR)

### DETAILED DESCRIPTION OF THE PREFERRED EMBODIMENTS

Unexpectedly, it has been found, as described in the examples herein below, that halofuginone can inhibit the pathological process of renal fibrosis in vivo, possibly by inhibiting collagen type I synthesis, although another mechanisms could also be responsible. Indeed, irrespective of the specific mechanism, the data presented below clearly demonstrate the efficacy of halofuginone in inhibiting the pathological progression of renal fibrosis in vivo.

The present invention provides a composition for treating renal fibrosis, comprising a pharmaceutically effective amount of a compound in combination with a pharmaceutically acceptable carrier, the compound being a member of a group having the general formula:

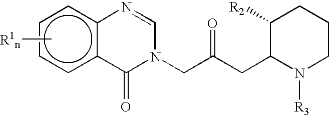

wherein: n=1-2

R₁ is a member of the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl and pharmaceutically acceptable salts thereof.

According to further preferred embodiments of the present invention, the compound is preferably halofuginone.

According to another embodiment the present invention provides a method of manufacturing a medicament for treating renal fibrosis, including the step of placing a pharmaceutically effective amount of a compound in a pharmaceutically acceptable carrier, the compound being a member of a group having the general formula:

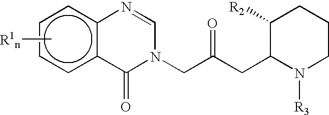

wherein: n=1-2

R₁ is a member of the group consisting of hydrogen, halogen, nitro, benzo, lower alkyl, phenyl and lower alkoxy;

R₂ is a member of the group consisting of hydroxy, acetoxy and lower alkoxy; and R₃ is a member of the group consisting of hydrogen and lower alkenoxy-carbonyl and pharmaceutically acceptable salts thereof.

According to yet another embodiment the present invention provides a method for the treatment of renal fibrosis in a subject, including the step of administering a pharmaceutically effective amount of a compound having the general formula:

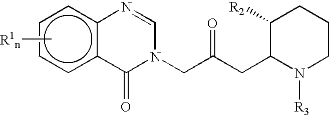

wherein: n=1-2

R₁ is a member of the group consisting of hydrogen, halogen, nitro, benzo, lower alkyl, phenyl and lower alkoxy;

R₂ is a member of the group consisting of hydroxy, acetoxy and lower alkoxy; and R₃ is a member of the group consisting of hydrogen and lower alkenoxy-carbonyl, and pharmaceutically acceptable salts thereof.

The renal fibrosis can be primary or secondary. The secondary condition may be caused by high hypertension, diabetes complications, autoimmune disease, and other underlying disorders and conditions.

According to further preferred embodiments of the present invention, the compound is preferably halofuginone. Hereinafter, the term “halofuginone” is defined as a compound having the formula:

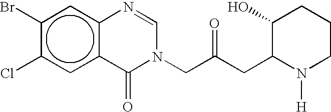

and pharmaceutically acceptable salts thereof. The composition preferably includes a pharmaceutically acceptable carrier for the compound.

Hereinafter, the term “subject” refers to a human or animal to whom halofuginone was administered. The term “patient” refers to human subjects. The term “treatment” includes both substantially preventing the process of renal fibrosis from starting and slowing or halting the progression of renal fibrosis once it has arisen. The term “renal fibrosis” refers to any fibrotic condition in the kidneys of the subject.

Hereinafter, the term “oral administration” includes, but is not limited to, administration by mouth for absorption through the gastrointestinal tract, buccal administration and sublingual administration. Compositions for oral administration include powders or granules, suspensions or solutions in water or non-aqueous media, sachets, capsules or tablets. Thickeners, diluents, flavorings, dispersing aids, emulsifiers, binders or preservatives may be desirable.

The term “parenteral administration” includes, but is not limited to, administration by intravenous drip or bolus injection, subcutaneous, or intra muscular injection. Formulations for parenteral administration may include but are not limited to sterile aqueous solutions which may also contain buffers, diluents and other suitable additives.

Although the specific quinazolinone derivative “halofuginone” is referred to throughout the specification, it is understood that other quinazolinone derivatives may be used in its place, these derivatives having the general formula:

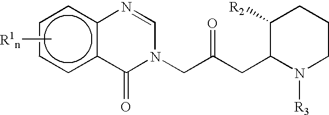

wherein: n=1-2

R₁ is a member of the group consisting of hydrogen, halogen, nitro, benzo, lower alkyl, phenyl and lower alkoxy;

R₂ is a member of the group consisting of hydroxy, acetoxy and lower alkoxy; and R₃ is a member of the group consisting of hydrogen and lower alkenoxy-carbonyl, and pharmaceutically acceptable salts thereof.

Compounds which are intended for the inhibition of renal fibrosis must be tested by an in vivo model for their ability to slow or halt the pathological process leading to deposition of fibrotic tissue.

Such experiments were conducted for the collagen type I synthesis inhibitor halofuginone, as described in greater detail in the Examples below. Renal fibrosis has been induced in rats that undergo renal mass reduction (RMR) or sham operation. The present invention may be more readily understood with reference to the following illustrative examples and figures.

### EXAMPLES

While the invention will now be described in connection with certain preferred embodiments in the following figures and examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following figures and examples which include preferred embodiments will serve to illustrate the practice of this invention, it being understood that the particulars shown are by way of example and for purposes of illustrative discussion of preferred embodiments of the present invention only, and are presented in the cause of providing what is believed to be the most useful and readily understood description of formulation procedures as well as of the principles and conceptual aspects of the invention.

### Example 1

A solution of halofuginone was prepared by dissolution of powder of halofuginone hydrobromide in aqueous media containing suitable buffer. Male Wistar rats (weighing 300±30 g at the start of the experiment) were used in this study after being allowed to acclimatize to their environment for one week. Rats were assigned to undergo renal mass reduction (RMR) by 5/6 nephrectomy or sham operation, under anesthesia with intraperitoneal injection of pentobarbital (35 mg/kg body weight). RMR was performed by ligature of 2 of 3 major branches of the left renal artery and right nephrectomy in the same session. Sham rats undergo exposure of the kidneys and removal of the peri-renal fat, without undergoing RMR. After 24 hours recovery of the rats were assigned to one of the following groups:

1) Group I: RMR rats, oral gavage with halofuginone 0.2 mg/kg/day started 24 hours post surgery.

2) Group II: RMR rats, oral gavage with normal saline daily, started 24 hours post surgery.

3) Group III: age matched, sham operated rats served as the controls.

All animals were allowed free access to a standard diet and water ad libitum. At sacrifice (10 weeks after RMR), kidneys were removed and processed for in situ hybridization, immunohistochemistry and histological evaluation.

Light microscopy studies: specimens were fixed in 10% buffered formaldehyde and embedded in paraffin. Histological sections of 4-5μ thickness were stained with hematoxylin-eosin (HES), periodic acid Schiff (PAS) and Masson trichrome (light green). A semi-quantitative score was used to evaluate the degree of glomerulosclerosis, mesangial expansion and proliferation and tubulo-interstitial changes. A minimum of 30 glomeruli in each specimen was examined and the severity of the lesions was graded from 0 to 4+, according to the percentage of glomerular involvement. Thus, a 1+ lesion represented 25% of the glomeruli and 4+ lesion indicated that 100% of the glomeruli were involved. An injury score was obtained by multiplying the degree of damage (0-4+) by the percentage of glomeruli with the same degree of lesions. The evaluation of tubulointerstitial fibrosis was performed with the point-counting method using a Zeiss I integrating eyepiece.

There was a significant decrease in tubulointerstitial fibrosis in halofuginone treated rats compared to the control group. The presence of glomerulosclerosis and mesangial proliferation was also less accentuated in halofuginone-treated rats (Table 1). These results show that rats treated with halofuginone, even at a low dose, exhibited better preservation of renal function.

TABLE 1
THE EFFECT OF HALOFUGINONE ON 5/6
NEPHRECTOMY IN RATS:
LIGHT MICROSCOPY (PRELIMINARY RESULTS)

| | Glomerulus | | | Interstitium | |
|---|---|---|---|---|---|
| | Prolif- | | | | Infil- |
| | eration | Sclerosis | Tubules | Fibrosis | tration |
| Halofuginone | | | | | |
| Group I | | | | | |
| (Rat #) | | | | | |
| 1 | Mild | 0 | Normal | − | − |
| 2 | Moderate | 0 | Few atrophic | + | + |
| 3 | Mild | 0 | Few atrophic | − | − |
| 4 | Mild | 0 | Few atrophic | − | − |
| 5 | Mild | 0 | Few atrophic | − | + |
| 6 | Moderate | 0 | Few atrophic | + | ++ |
| Control | | | | | |
| Group II | | | | | |
| (Rat #) | | | | | |
| 1 | Severe | 1 | Atrophic ++ | ++ | + |
| 2 | Severe | 1 | Dilated ++ | − | + |
| 3 | Severe | 1 | Dilated − | ++ | ++ |
| | | | Atrophic ++ | | |
| 4 | Severe | 1 | Atrophic ++ | ++ | + |
| 5 | Moderate | 1 | Atrophic + | ++ | ++ |
| | to | | | | |
| | severe | | | | |

### Example 2

Male Wistar rats (weighing 300±30 g at the start of the experiment) were used in this study. They were allowed to acclimatize to their environment for one week. Rats were assigned to undergo renal mass reduction (RMR) by 5/6 nephrectomy or Sham operation, under anesthesia with intraperitoneal injection of pentobarbital (35 mg/kg body weight). RMR was performed by ligature of 2 of 3 major branches of the left renal artery and right nephrectomy in the same session. Sham rats have undergone exposition of the kidneys and removal of the peri-renal fat. After 24 hours recovery the rats were assigned to one of the following groups:

1) Group I: RMR rats, oral gavage with halofuginone 0.2 mg/kg/day started 24 hours post surgery.

2) Group II: RMR rats, oral gavage with normal saline daily, started 24 hours post surgery.

3) Group III: age matched, sham operated rats served as the controls.

All animals were allowed free access to a standard diet and water ad libitum. Every week, systolic blood pressure was measured by tail cuff manometry and urine samples were collected individually in metabolic cages for determination of total protein and creatinine excretion. Protein concentration in urine was determined by a colorimetric method using pyrogallol-red molybdate complex (cobas integra 700, Roche). Body weight was also measured. At sacrifice (10 weeks after RMR) blood was withdrawn from abdominal aorta for determination of creatinine and halofuginone concentrations. Serum creatinine was measured with a Hitachi model 747 autoanalyzer, using the kinetic Jaffe method.

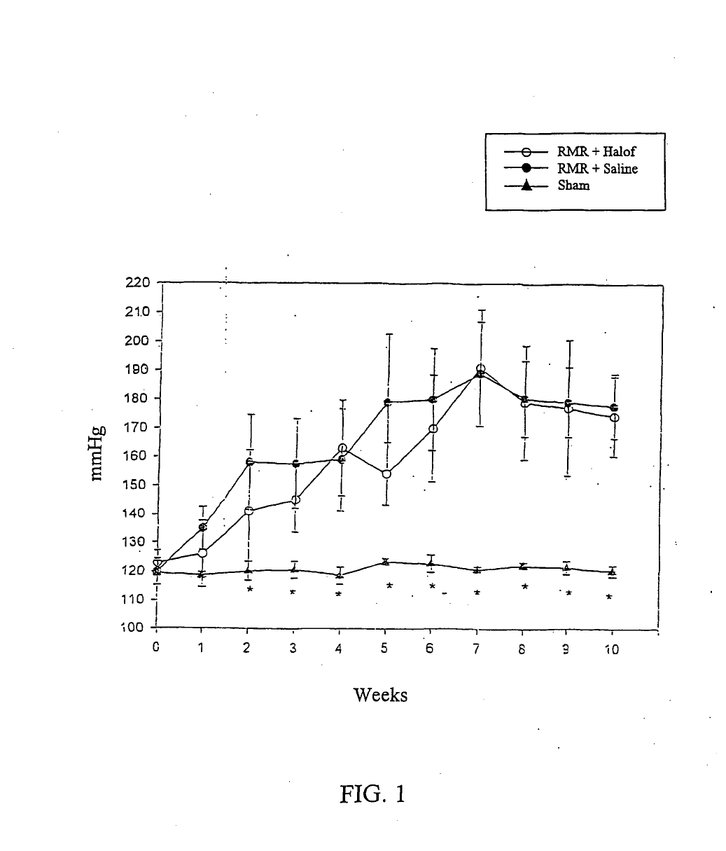

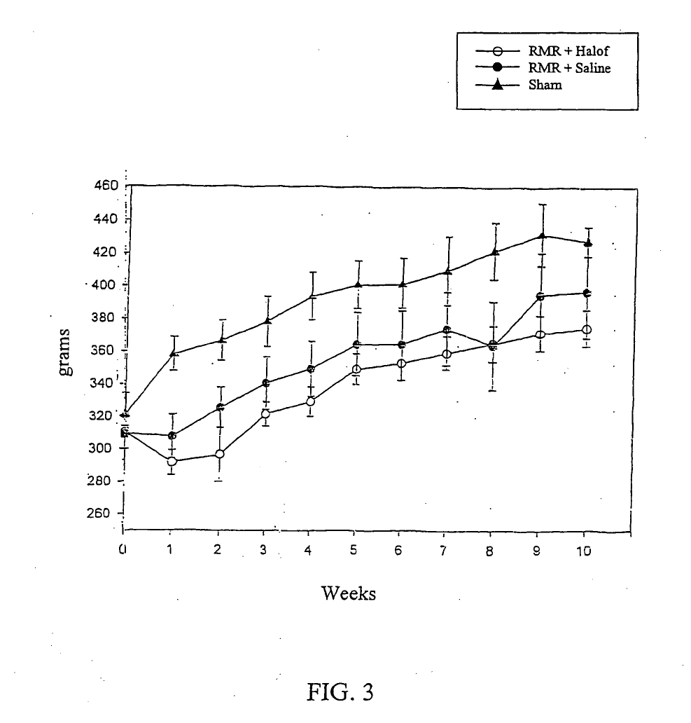

After a small decrease in body weight at the end of the first week in both nephrectomized groups, body weight increase was similar in halofuginone treated and control groups (FIG. 3), suggesting that food intake was similar in both groups throughout the time of the experiment. The two nephrectomized groups showed also no significant difference in systolic blood pressure, which increased progressively reaching a peak at 7 weeks (FIG. 1). No significant variation from baseline level was noted in the SHAM operated group. These results demonstrate the adequacy of the model undertaken for evaluating the efficacy of renoprotective action of halofuginone.

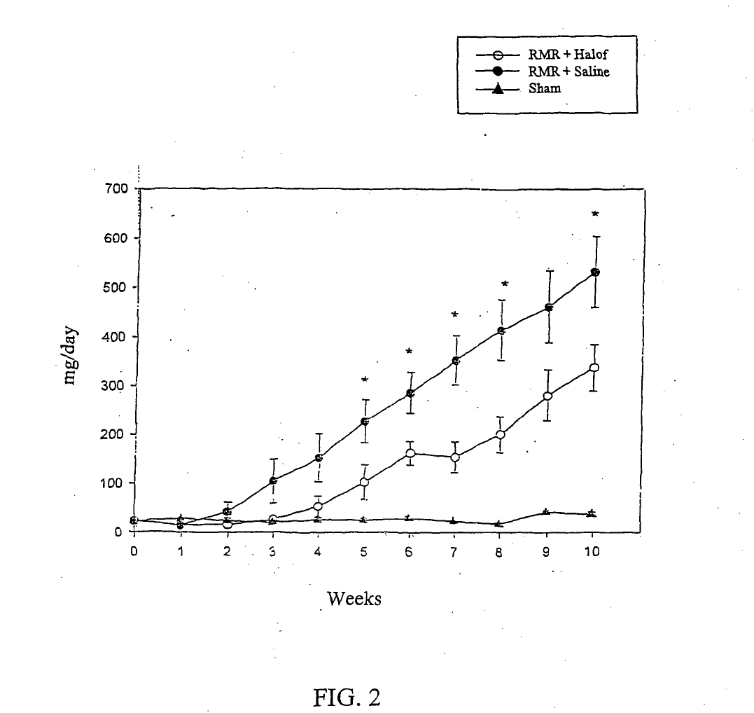

As shown in FIG. 2, rats treated with halofuginone had lower levels of proteinuria than control nephrectomized group. This difference was statistically significant from week 5 post—nephrectomy and on.

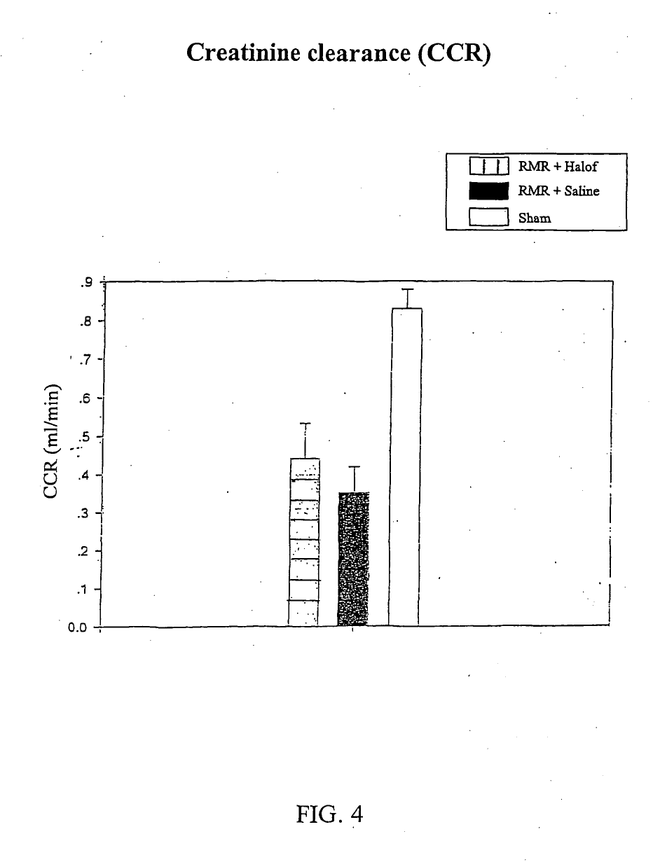

As expected, CCR was lower in RMR groups when compared to Sham rats. CCR was higher in the group treated with halofuginone at the end of study (0.44+0.09 vs 0.35+0.07 nl/min, p=0.06, FIG. 4)

These results show that halofuginone have a beneficial effect on the kidneys, delaying the proteinuria as well as reducing the deterioration of creatinine clearance. Both phenomena suggest preservation of renal function.

### Example 3

Method of Treatment of Renal Fibrosis

As noted above, halofuginone has been shown to be an effective inhibitor of renal fibrosis. The following example is an illustration only of a method of treating renal fibrosis with halofuginone, and is not intended to be limiting.

The method includes the step of administering halofuginone, in a pharmaceutically acceptable carrier as described above, to a subject to be treated. Halofuginone is administered according to an effective dosing methodology, preferably until a predefined endpoint is reached, such as the absence of further progression of renal fibrosis in the subject, the inhibition of renal fibrosis or the prevention of the formation of renal fibrosis.

Halofuginone can be administered to a subject in a number of ways, which are well known in the art. Hereinafter, the term “subject” refers to a human or animal to whom halofuginone was administered. For example, administration may be done orally, or parenterally, for example by intravenous drip or bolus injection, subcutaneous, or intramuscular injection.

Compositions for oral administration include powders or granules, suspensions or solutions in water or non-aqueous media, sachets, capsules or tablets. Thickeners, diluents, flavorings, dispersing aids, emulsifiers, preservatives or binders may be desirable.

Formulations for parenteral administration may include but are not limited to sterile aqueous solutions which may also contain buffers, diluents and other suitable additives.

Dosing is dependent on the severity of the symptoms and on the responsiveness of the subject to halofuginone. The attending physician can easily determine optimum dosages, dosing methodologies and repetition rates.

### Example 4

Method of Manufacture of a Medicament Containing Halofuginone

The following is an example of a method of manufacturing halofuginone. First, halofuginone is synthesized in accordance with good pharmaceutical manufacturing practice. Examples of methods of synthesizing halofuginone, and related quinazolinone derivatives, are given in U.S. Pat. No. 3,338,909. Next, halofuginone is placed in a suitable pharmaceutical carrier, as described in Example 3 above, again in accordance with good pharmaceutical manufacturing practice.

What is claimed is:

1. A composition for treating renal fibrosis, comprising a pharmaceutically effective amount of a compound in combination with a pharmaceutically acceptable carriers, said compound being a member of a group having the general formula: 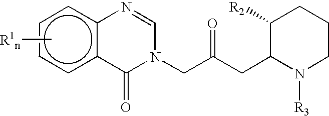
wherein: n=1-2
R₁ is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof.

2. The composition of claim 1, wherein said compound is halofuginone.

3. The composition of claim 1 wherein said pharmaceutically acceptable carrier enables administration of the composition orally or parenterally in form of powder, granules, suspensions or solutions in water or non aqueous media, sachets, capsules or tablets.

4. A method for treating renal fibrosis in a subject, comprising administering to said subject a therapeutically effective amount of a pharmaceutical composition comprising as an active ingredient a compound having the general formula: 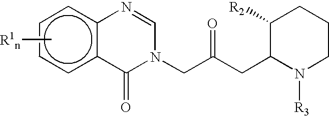
wherein: n=1-2
R₁ is a member of the group consisting of hydrogen, halogen, nitro, benzo, lower alkyl, phenyl and lower alkoxy;
R₂ is a member of the group consisting of hydroxy, acetoxy and lower alkoxy, and
R₃ is a member of the group consisting of hydrogen and lower alkenoxy-carbonyl, and pharmaceutically acceptable salts thereof.

5. The method of claim 4, wherein said compound is halofuginone.

6. The method of claim 4, wherein said pharmaceutical composition is suitable for administration orally or parenterally in the form of powder, granules, suspensions or solutions in water or non aqueous media, sachets, capsules or tablets.

7. The method of claim 4, wherein the renal fibrosis condition is primary or secondary.

8. The method of claim 7 wherein the secondary condition is caused by hypertension, diabetic complications, or autoimmune diseases.

9. A method for preventing renal fibrosis from progressing to end-stage renal failure comprising administering to a subject in need thereof a therapeutically effective amount of compound in a pharmaceutically acceptable carrier, said compound being a member of a group having the general formula: 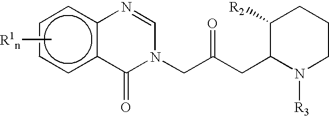
wherein: n=1-2
R₁ is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof.

10. The method of claim 9, wherein said compound is halofuginone.

11. The method of claim 9, wherein said pharmaceutically acceptable carrier enables administration of the composition orally or parenterally in the form of powder, granules, suspensions or solutions in water or non aqueous media, sachets, capsules or tablets.

12. A method for preparing a pharmaceutical composition for treating renal fibrosis which comprises combining a compound being a member of the group having the general formula: 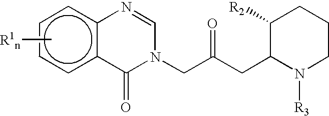
wherein: n=1-2
R₁ is a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof, with a pharmaceutically acceptable carrier to form the composition for preparing a pharmaceutical composition for treating renal fibrosis.

13. The method of claim 12, wherein the compound is halofuginone.

14. The method of claim 12, wherein said medicament is suitable for administration orally or parenterally in the form of powder, granules, suspensions or solutions in water or non aqueous media, sachets, capsules or tablets.
